(19) United States
(12) Patent Application Publication
(10) Pub. No.: US 2020/0001100 A1
(43) Pub. Date: Jan. 2, 2020

(54) ANTALGIC MAT FOR MULTIPLE AND MAGNETIC THERAPY

(71) Applicant: Salvatore SORTINO, Rosolini (SR) (IT)

(72) Inventor: Salvatore SORTINO, Rosolini (SR) (IT)

(21) Appl. No.: 16/487,664

(22) PCT Filed: Feb. 23, 2018
(86) PCT No.: PCT/IB2018/051151

§ 371 Date: Aug. 21, 2019

(30) Foreign Application Priority Data
Feb. 23, 2017 (IT) 202017000020814

(51) Int. Cl. A61N 2/00; A61K 36/02; A61K 9/70; D06N 7/00

(52) U.S. Cl. CPC A61N 2/008; A61N 2/002; A61K 36/02; D06N 2201/082; D06N 7/00; D06N 2201/085; A61K 9/7007

(57) ABSTRACT

A pain-relieving mat for multiple and magnetic therapy is composed of a tissue of bioceramic fibre, magnetic bands of north-south magnetization, central magnetic bands, a lower tissue, configured to allow connecting the mat to any generator device for magnetotherapy, and engineered yarns, such as copper fibre, carbon fibre, silver fibre.

### APPLICATION FIELD OF THE INVENTION

The present finding belongs to the field of the treatment of inflammations and physical rebalancing of a person by a series of embodiments, including magnetotherapy.

### STATE OF THE ART

Magnetotherapy is one of the physical therapies with the least number of contraindications, as indicated also by a number of clinical studies carried out with electromagnetic waves.

Magnetotherapy acts at the level of the single cells in the body, “recharging” them with energy.

Living cells, whether nerve or muscle cells, and generally all the cells in a body, have an electric potential, referred to as the electric resting membrane potential (Em), which is calculated by the difference of intra- and extra-cellular electric charge. As a function of the cell type, such value ranges from −55 to −90 mV.

The lower this potential differential is, the lower the cell vitality is. Generally, after a muscle injury, trauma, wound, disease, o other adverse events, the cells surrounding the affected area loose part of their electric charge (i.e., they change their differential potential).

Generally, magnetotherapy requires medium to long treatment times. Typically, it is recommended to carry out cycles of 45-60 days for a minimal duration of 2-3 hours a day.

Nonetheless, there are no risks of overdosing, thus it is also possible to carry out a night therapy during several hours without worry.

### DISCLOSURE AND ADVANTAGES OF THE FINDING

An object of the present invention is to provide to the art a mat for magnetotherapy treatment in the context of a solution which is simple, reasonable, and quite cost-efficient.

It is noted that the term “mat” used herein should encompass all those devices, such as small mats, blankets, mattress covers, obtained according to the teachings described herein below.

Such and other objects are achieved by virtue of the characteristics of the invention set forth in the independent claim 1. The dependant claims outline preferred and/or particularly advantageous aspects of the invention.

The set of technologies, which are applied to the mat, make the mat a unique object, by virtue of the series of benefits that are achieved by the use thereof. In fact, the mat according to the present invention allows simultaneously acquiring several benefits with only one session, thus in considerably reduced times compared to the known solutions.

Said objects and advantages are all achieved by the mat for magnetotherapy that is the subject-matter of the present finding, which is characterized by what is provided for in the claims set forth below.

### DESCRIPTION OF THE FINDING

The mat which is the subject-matter of the finding is, during use, rested and/or laid on a mattress and possibly secured by means of angular elastics that can be provided for and secured to the mat.

The mat comprises a suitable jack that can be connected in an operative configuration with a magnetotherapy apparatus, i.e., a low or/and high frequency wave generator.

Once the apparatus is activated, it is started. The duration of the applications varies based on the disease and the chronicity thereof (at least 30 minutes a day to a complete 6-hour cycle overnight).

A wire exits the mat, ending in a small metal ball: the energy built up during the das is thus discharged by virtue of metal ball, which is dropped to the ground, where it contacts the floor, in an operative configuration.

By virtue of such solution, the static energy built up during the das is discharged; furthermore, by virtue of the magnetotherapy, a benefit is obtained, resulting from the natural re-polarization of the body during night-time rest.

The mat is obtained with a highly innovative and engineered tissue.

In more detail, the mat is made of a first bioceramic fibre tissue. By way of example, the bioceramic tissue is a tissue to which bioceramic material is added, i.e., biocompatible ceramic materials of various kinds. Magnetic bands for magnetization, specifically, north-south bands. In an embodiment, the magnetic bands are located at the opposite ends of the mat so as to be positioned, during operation, two facing the feet side, and two facing the head side. In other words, the two pairs of bands will be located on longitudinally opposite ends, where longitudinal direction means the direction defined by the head-feet direction of the user.

In addition to the magnetization bands, also central magnetic bands are present. Such magnetic bands are preferably located centrally with respect to the length of the mat, i.e., to the above-mentioned longitudinal direction.

Preferably, the mat comprises at least three central magnetic bands.

According to a preferred embodiment, the central magnetic bands are interposed between the magnetization bands located at the feet side and the head side.

A lower (end) tissue is also present, which is referred to as a “super contact” in the jargon; in other words, it is configured so as to allow connecting the mat to any generator device (low-high frequency) for magnetotherapy.

Engineered yarns are further present, such as, for example, copper fibre, carbon fibre, silver fibre, which are suitably arranged as set forth herein below.

The mat may also comprise a ground discharge system.

The above-mentioned bioceramic fibre tissue is characterized by its mirror function. Each human being emits his/her specific FIR (Far Infra-Red Ray) wave, which is crucial for his/her life processes. In other words, the mirror technology is capable of reflecting for each single individual, his/her own FIR energy waves.

A preferred embodiment provides that the tissue of the mat is composed of at least two different bioceramic fibres. Thanks to this solution, a pain-relieving effect is achieved.

As regards the arrangement of the above-mentioned engineered yarns, it is pointed out that, along at least one edge of the mat, a carbon double wire is present, which is suitable to protect the body against radiations emitted by the electrical equipment with which we are in contact during most of our daily life, contributing to a healthy and pleasant rest.

In an embodiment, as regards the arrangement of the above-mentioned engineered yarns, it is pointed out that the upper side of the mat is made of silver fibre, preferably a SILVER LIFE tissue, i.e., a fibre treated with silver ions, (antimicrobial and disinfectant, as well as odour inhibiting properties).

According to another aspect, copper filaments are present in for the complete discharge of the electrostatic energy. The copper or silver wire or tissue is connectable to a polo of a low-high frequency generator. By virtue of such connection, a radio magnetic monopolar field is generated on the tissue, which is usually emitted in a homogeneous manner onto the entire area of interest, or individually or simultaneously with nuclei to generate a low frequency.

The bioceramic technology, with its reflecting ability, recovers the FIR waves emitted by the human body and allows a reintegration by reflecting thereof, and thus to the benefit of arthritic muscular pathologies.

The magnetic technology has the advantage the, through the provision of a north-south polarization, the user does not have to worry about the bed positioning, thus deriving all the possible benefit with the right polar positioning, also hindering the residual radiations and creating a psychophysical balance during sleep.

In an embodiment, the use of magnetic bases with a stable low-intensity field is provided, which concurs to recharge the cells, thus limiting necrosis, hence preventing and creating an antiinflammatory and analgesic action.

In an embodiment, the application of a tissue having a conductive layer and/or wrapped layers for the emission of pulsed electromagnetic waves both at low frequency and high frequency (for a prevention and rebalance) is provided. Such feature allows improving the circulation, resulting in an integral benefit to the user's body, not only at the joint level, but also at the vascular level, moreover regenerating endorphins and melanin to stabilize the brain functions, thus reducing insomnia as well as stress. In addition, one can benefit from the reconditioning and energy generating function, to the benefit of the cellular recharge which thus acts on arthritic, muscular, vascular, and regenerative pathologies.

By the application of the ground discharge system, it is possible to discharge all the static energy built up during the several, challenging everyday activities and, with the suitably applied technology, thus promoting a complete relaxation to the entire body.

In an embodiment, carbon engineered yarn is used on the side parts of the mat, which protects the body against radiations emitted by electrical accessories with which the user can be in contact during most of our everyday life, concurring to a healthy, pleasant rest.

As mentioned above, in the upper part of the mat, the use of a fibre treated with silver ions allows interacting against microbes and bacteria, while providing pest control and disinfection, as well as blocking the absorption of malodours.

The mat can further employ, in addition to what has been stated above, a further tissue composed at least partially of fibres obtained from seaweed extract. The advantage of the seaweed content is the set of vitamins A, B, E, further minerals, such as magnesium, calcium, potassium, and phosphorus, amino acids and microelements that last throughout the tissue life for a benefit to the body and the skin. This has an antioxidant, antiinflammatory, revitalizing and skin protective effect, neutralizes most of the free radicals, referred to as RNS and ROS, which are produced as a function of the circumstances affecting us, such as stresses, improper nutrition, pollution, etc. By virtue of these aspects, further benefits are obtained, such as a slowing down of aging and a reduction of diseases such as atopic dermatites, etc.

1. A pain-relieving mat for multiple and magnetic therapy, comprising at least:
a. a first tissue composed of at least one bioceramic fibre,
b. two pairs of magnetic bands for a north-south magnetization located at the ends of said mat, so as to be arranged, during use, two at the feet side and two at the head side,
c. central magnetic bands, with respect to the length of the mat,
d. a lower tissue, configured to:
i. allow connecting the mat to a generator device for magnetotherapy, configured to generate high and/or low frequency electromagnetic waves in,
ii. generate a monopolar radio magnetic field on the tissue and which, in an operative configuration, is regularly emitted in a homogeneous manner on the entire area of interest, or singularly or simultaneously with nuclei to generate a low frequency.
e. one or more engineered yarns, such as copper fibre, carbon fibre, silver fibre.

2. The mat according to claim 1, wherein a wire exits, which ends in a small metal ball that is configured to contact the floor, in an operative configuration, discharging the energy built up during the day.

3. The mat according to claim 1, wherein along at least one edge it comprises a carbon double wire.

4. The mat according to claim 1, wherein the upper side of the mat is made of silver fibre.

5. The mat according to claim 1, wherein copper filaments for the complete discharge of the electrostatic energy are present within the first tissue.

6. The mat according to claim 1, which further comprises a suitable jack connectable, in an operative configuration, to a magnetotherapy apparatus and/or a low-high frequency wave generator.

7. The mat according to claim 1, further comprising a further tissue obtained from the seaweed extract product with an antioxidant, anti-inflammatory, revitalizing and skin protective effect, neutralizes the free radicals, referred to as RNS and ROS, which are produced as a function of circumstances such as stresses, improper nutrition, pollution, etc.

8. The mat according to claim 1, wherein the central magnetic bands, with respect to the length of the mat, are three.

9. The mat according to claim 1 wherein the tissue is composed of at least two different bioceramic fibres configured to generate a pain-relieving effect.

10. The mat according to claim 1, wherein said one or more engineered yarns for the upper side of the mat intended to be in contact with the user.
